(19) United States
(12) Patent Application Publication
(10) Pub. No.: US 2011/0118852 A1
(43) Pub. Date: May 19, 2011

(54) PIEZOELECTRIC IMPLANT

(75) Inventor: David E. Evans, West Chester, PA (US)

(73) Assignee: SYNTHES USA, LLC, West Chester, PA (US)

(21) Appl. No.: 12/949,121

(22) Filed: Nov. 18, 2010

Related U.S. Application Data
(60) Provisional application No. 61/262,346, filed on Nov. 18, 2009.

(51) Int. Cl. A61F 2/02

(52) U.S. Cl. 623/24

(57) ABSTRACT

A piezoelectric implant configured to stimulate bone growth in surrounding bone, tissues, and/or bodily fluids. Anatomical loading of the piezoelectric implant causes the piezoelectric implant or components thereof to emit a signal, such as an electric current and/or electromagnetic field that promotes and/or enhances osteoblastic activity in the surrounding bone, tissues, and/or bodily fluids, thereby enhancing bone remodeling and/or fusion. The piezoelectric implant can be configured with a variety of piezoelectric components, such as individual piezoelectric elements or piezoelectric frames, and/or conductors configured to conduct the signal between the piezoelectric components and the surrounding bone, tissues, and/or bodily fluids.

### CROSS-REFERENCE TO RELATED APPLICATIONS

This patent application claims priority to U.S. provisional patent application number 61/262,346, filed Nov. 18, 2009, which is incorporated herein by reference in its entirety.

### BACKGROUND

A key contributor to the degree of success achieved with a spinal fusion surgery is the ability of the patient's body to successfully remodel the bone necessary for fusion to succeed. For example, if a patient's body is unable to remodel the bone necessary for fusion to succeed before the fatigue limit of the implant is realized, the strength and effectiveness of the implant may not prevent its eventual failure. Therefore, the stability of an implant and its associated anatomical structure can be improved if the bone remodels itself quickly. Moreover, a patient typically encounters less mechanically induced pain when the bone remodels and fuses quickly.

A known method for enhancing bone remodeling involves the application of bone morphogenic proteins (BMP) to implants and/or surrounding bony surfaces. However, the use of BMP can be prohibitively expensive, and the use of BMP in some instances has been associated with bone growth beyond preferred levels in some patients. Another technique for enhancing bone remodeling is via electrical stimulation. Examples of electrical stimulation techniques include an implantable direct current (DC) device, capacitive coupling, and pulsed electromagnetic field (PEMF) generators. However, implantable DC devices typically involve multiple surgeries over a short period of time. For instance, a DC device may be implanted surgically and subsequently removed surgically within a span of as little as six months. Capacitive coupling techniques typically require cast immobilization for effective application. Additionally, the electrode pads typically associated with capacitive coupling techniques have been known to require weekly re-application of gel and the battery in capacitive coupling devices may require daily replacement. Thus, patient compliance with capacitive coupling techniques typically presents a substantial challenge. When treatment is provided via a PEMF device, the electromagnetic field is typically applied to the desired area via an obtrusive external device worn by a patient. However, because optimum PEMF dosage is believed to be on the order of ten hours per day, patient compliance typically presents a substantial challenge to the success of this technique as well.

### SUMMARY

In accordance with one embodiment, a piezoelectric implant assembly includes an implant body extending along a transverse direction between opposing upper and lower bone-facing surfaces and a piezoelectric component embedded in the implant body such that the piezoelectric component does not contact surrounding bone. When the implant body is subjected to an anatomical load, the piezoelectric component emits a signal that stimulates growth in the surrounding bone.

### BRIEF DESCRIPTION OF THE DRAWINGS

The foregoing summary, as well as the following detailed description of the preferred embodiments of the application, will be better understood when read in conjunction with the appended drawings. For the purposes of illustrating the piezoelectric implant assemblies of the instant application, there are shown in the drawings preferred embodiments. It should be understood, however, that the instant application is not limited to the precise arrangements and/or instrumentalities illustrated in the drawings, in which:

FIG. 1A is a perspective view of a piezoelectric implant assembly constructed in accordance with an embodiment;

FIG. 1B is a sectional perspective view of the piezoelectric implant assembly illustrated in FIG. 1A;

FIG. 1C is an exploded perspective view of the piezoelectric implant assembly illustrated in FIG. 1A;

FIG. 1D is a front elevation view of the piezoelectric implant illustrated in FIGS. 1A-C inserted into an intervertebral space.

FIG. 2A is a perspective view of a piezoelectric frame constructed in accordance with an embodiment;

FIG. 2B is a perspective view of a piezoelectric frame constructed in accordance with another embodiment;

FIG. 3A is a perspective view of a piezoelectric implant assembly constructed with the implant frame illustrated in FIG. 2A, in accordance with an embodiment;

FIG. 3B is a perspective view of the piezoelectric implant illustrated in FIG. 3A constructed in accordance with an alternative embodiment;

FIG. 3C is a perspective view of the piezoelectric implant illustrated in FIG. 3A constructed in accordance with another alternative embodiment.

### DETAILED DESCRIPTION

For convenience, the same or equivalent elements in the various embodiments illustrated in the drawings have been identified with the same reference numerals. Certain terminology is used in the following description for convenience only and is not limiting. The words “right”, “left”, “upper” and “lower” designate directions in the drawings to which reference is made. The words “inward”, “inwardly”, “outward”, and “outwardly” refer to directions toward and away from, respectively, the geometric center of the device and designated parts thereof. The words, “anterior”, “posterior”, “superior”, “inferior”, “lateral”, “medial”, “sagittal”, “axial”, “coronal,” “cranial,” “caudal” and related words and/or phrases designate preferred positions and orientations in the human body to which reference is made and are not meant to be limiting. The words “vertebral body” as used herein should be interpreted broadly to include all the bones and bony structures found within and in the immediate proximity of the human spinal system, including but not limited to those found in the cervical region, the thoracic region, the lumbar region, and the sacral curve region. The terminology intended to be non-limiting includes the above-listed words, derivatives thereof and words of similar import.

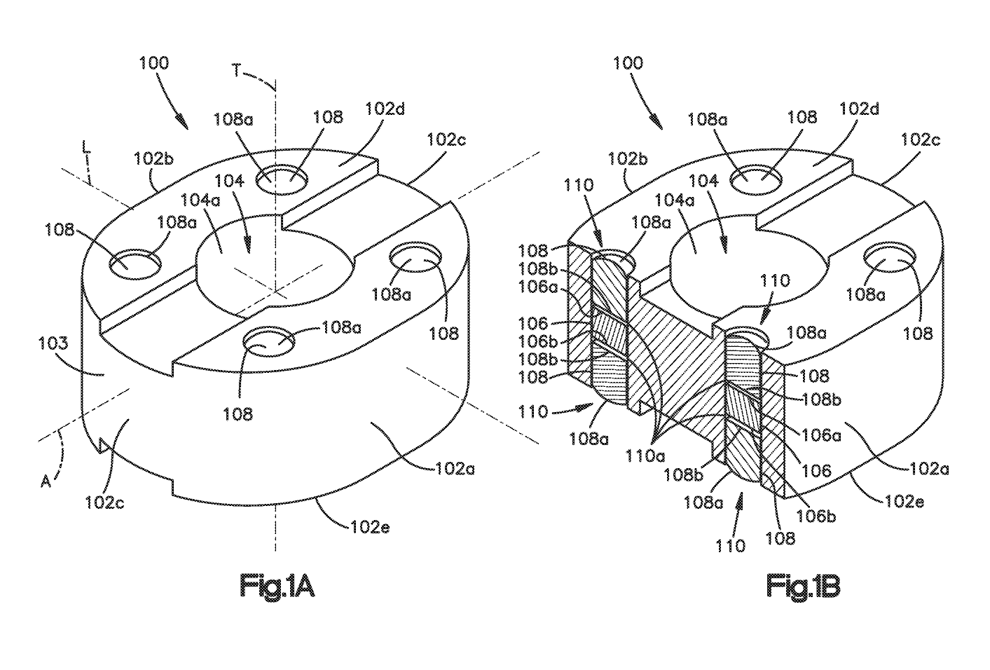

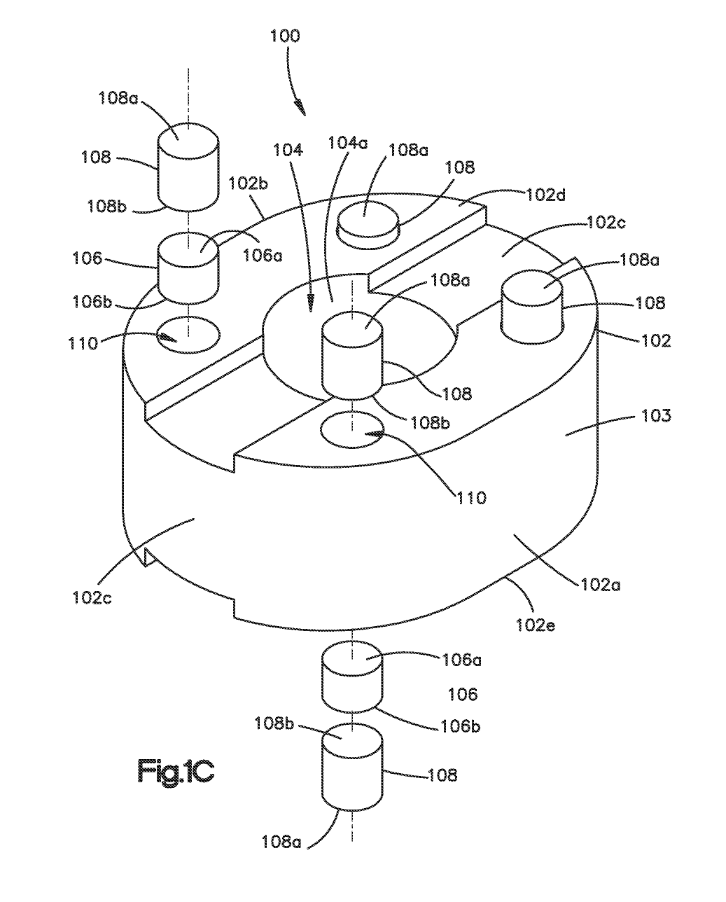

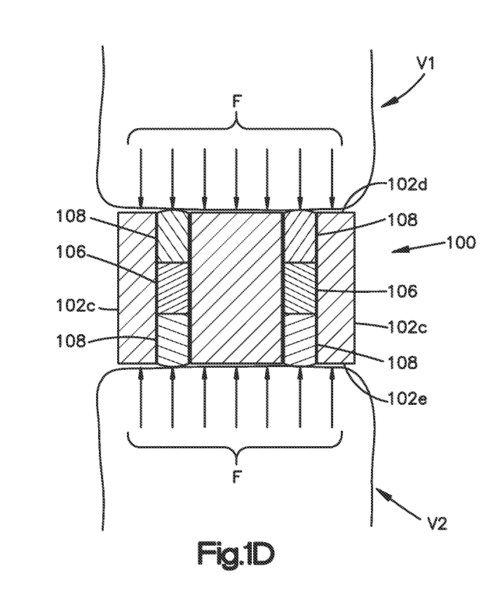

Referring now to FIGS. 1A-D, a piezoelectric implant in the form of a piezoelectric implant assembly 100 is illustrated. The implant assembly 100 includes an implant body 102 having a front, or anterior side 102a and an opposing rear, or posterior side 102b, opposing sides 102c, the anterior and posterior sides 102a-b and the sides 102c together defining an outer, or side surface 103 extending around the entirety of the implant body 102. The implant body 102 extends between an upper bone-facing surface 102d and a lower bone-facing surface 102e. All or a portion of the upper and lower bone-facing surfaces 102d-e may have gripping structure such as teeth, spikes, or similar structures, formed thereon, the gripping structure configured to facilitate gripping engagement between the upper and lower surfaces 102d-e and surrounding, or neighboring, bone, tissues, and/or bodily fluids, such as adjacent bony surfaces of the end plates of adjacent vertebral bodies V1 and V2.

The anterior and posterior sides 102a-b are spaced apart from each other along a longitudinal direction L. The sides 102c are spaced apart from each other along a lateral direction A that is angularly offset (e.g., perpendicular) with respect to the longitudinal direction L. The bone-facing surfaces 102d-e are spaced apart from each other along a transverse direction T that is angularly offset (e.g., perpendicular) with respect to both the longitudinal direction L and the lateral direction A. When the implant assembly 100 is inserted between adjacent bony surfaces, or surrounding bone, for example the endplates of adjacent vertebral bodies after a discectomy has been performed, the longitudinal direction L extends generally in an anterior-posterior direction, the lateral direction A extends generally in a medial-lateral direction, and the transverse direction T extends generally in a cranial-caudal direction. It should be appreciated that while the implant body 102 is illustrated as a generally rounded, rectangular shaped body, that any other body geometry can be used as desired.

The body 102 can have one or more bores, such as implant bore 104, extending from the upper bone-facing surface 102d through the lower bone-facing surface 102e. The implant bore 104 defines an inner bore surface 104a. The bores can be cylindrical in shape, such as implant bore 104, or can be of any other geometry as desired. Implant bore 104 can be filled with bone growth inducing substances, for example bone cement infused with piezoelectric crystals, allograft, or the like, to allow bony ingrowth and to assist in fusion between the implant assembly 100 and adjacent vertebral bodies. Implant bore 104 can be defined centrally with respect to the implant body 102, as illustrated, or in any other location within the body 102 as desired.

The implant body 102 further includes at least one component, or element, made of piezoelectric material, such as piezoelectric elements 106. The piezoelectric components are configured to generate, or emit signals, such as electric current or electromagnetic fields, in response to compressive forces, for example those generated by anatomical loading on the implant body 102, as described in more detail below. The piezoelectric elements 106 of the illustrated embodiment each have an upper, or superior surface 106a, and an opposing lower, or inferior surface 106b. The piezoelectric elements 106 are preferably embedded in the body 102 such that the piezoelectric elements 106 will not come into contact with surrounding, or neighboring, bones, tissues, and/or bodily fluids. Embedding the piezoelectric elements in the implant body 102 allows various electrical and/or electromagnetic effects to be induced into surrounding bone, tissues, and/or bodily fluids as described in more detail below. The piezoelectric elements 106 can be embedded in the implant body 102 in a number of ways. For example, as depicted in FIG. 1B, the piezoelectric elements 106 can be embedded within the body 102 at a location between the upper and lower bone-facing surfaces 102d-e, respectively. In operation, when an anatomical load is applied to the piezoelectric implant assembly 100, as described in more detail below, forces will be transferred from the implant body 102 to the piezoelectric elements 106. In response to the forces, the piezoelectric elements will generate, or emit a signal in the form of an electromagnetic field into surrounding bone, tissues, and/or bodily fluids.

Although the piezoelectric implant assembly 100 as illustrated includes four cylindrically shaped piezoelectric elements 106, any number and/or combination of shapes can be utilized for the piezoelectric elements 106 as desired. For example, one or more piezoelectric elements 106 can be partially or fully embedded within the implant body 102 as piezoelectric layers of the implant body 102. Alternatively, one or more piezoelectric elements 106 can be embedded within the implant body 102 as a piezoelectric filling, the piezoelectric filling disposed in one or more distinct, up to a continuous, channel defined within the implant body 102. The piezoelectric filling can be configured entirely of piezoelectric material, or can be a piezoelectric mixture, such as a non electrically conductive base material mixed with pieces of piezoelectric components as described in more detail below.

The piezoelectric elements 106 can be embedded into the implant body 102 by insertion, for instance into pre-drilled bores in the implant body 102, by overmolding the implant body 102 over the piezoelectric elements 106, or by any other desired embedding process. If the piezoelectric elements 106 are inserted, the piezoelectric elements 106 may be retained in position within the implant body 102 by a slide fit or press fit in the implant body, may be cemented to the implant body, or otherwise retained as desired. Insulation, potting material, and/or transmission components may be disposed between the outer surface of the piezoelectric element 106 and the implant body 102 as desired.

The piezoelectric elements 106 can be formed from single material or combination of materials such as, but not limited to, Berlinite, Tourmaline, Gallium orthophosphate, barium titanate, hydroxyapatite, apatite, sodium potassium niobate, quartz, lead zirconium titanate (PZT), or any other material that exhibits piezoelectric properties, or into which piezoelectric properties can be induced, as deemed design appropriate to the desired application, as well as a variety of crystalline-structured materials, including ceramic perovskite.

The implant body 102 of the piezoelectric implant assembly 100 may be formed from one or more materials such as polyetheretherketone (PEEK), including PEEK and/or porous PEEK combined with any other substance, Titanium, Carbon Fiber, ceramic, Polyethylene, Polycarbonate Urethane, Poly Methyl Methacralate (PMMA), stainless steel, diamond, quartz, Cobalt Chromium, Tantalum, allograft, autograft, xenograft, or any other implantable material. When the implant body 102 is constructed of a metal, an insulating element (not shown) can be included between the piezoelectric elements 106 and the implant body 102 in order to block the flow of current from the piezoelectric elements 106 into the implant body 102.

If it is desirable to conduct an electric current generated by the piezoelectric elements 106 directly into surrounding bone, tissues, and/or bodily fluids, a pathway for current flow between the embedded piezoelectric elements 106 and the surrounding bone, tissues, and/or bodily fluids can be established by disposing conducting elements, such as conductors 108, into the implant body 102. The conductors 108 include a body extending between an upper, or bone engaging end 108a and an opposing lower, or piezoelectric element engaging end 108b. The conductors 108 can be configured with body geometry, for example cross sectional geometry, that matches that of the piezoelectric elements 106, as illustrated, or can be configured with any other body geometry as desired. For example, the conductors 108 may be configured in the shape of balls, pins, spiked pins with or without shoulders, wires, leads of any shape, or the like, or can be configured in any combination of shapes as appropriate. Additionally, the conductors 108 can be configured to contact the surface of surrounding bone, to not contact any surrounding bone, or to be partially or completely embedded within surrounding bone. Moreover, it should be appreciated that electrical stimulation of surrounding bone, soft tissues, and/or bodily fluids may be achieved without the use of conductors 108. For example, a particular application of the piezoelectric implant assembly 100 might warrant direct contact between the piezoelectric element 106 and the bones, tissues, and/or bodily fluids intended to benefit from the electrical stimulation the piezoelectric implant assembly generates. The conductors 108 can further be configured to act as shielding, or insulating, conductors 108 that prevent direct contact between the piezoelectric elements 106 and surrounding bones, tissues, and/or bodily fluids, for instance when it is desirable to employ piezoelectric elements 106 that are non-biocompatible, such as those containing lead.

As illustrated in FIGS. 1A-D, the conductors 108 can be disposed into conductor bores 110 extending into the implant body 102 from the upper and/or lower bone facing surfaces 102d-e. The conductor bores 110 can be configured to be open at their interior ends 110a, such that the respective piezoelectric elements 106 are exposed, thereby providing contact surfaces for mating the conductors 108 to the piezoelectric elements 106 in conductive engagement. The conductors 108 can be constructed from any biocompatible conducting material, such as tantalum, or the like. If a piezoelectric element 106 is embedded between one or more of the conductors 108 (i.e., at least one conductor 108 is disposed above the respective piezoelectric element 106 and at least one conductor 108 is disposed below the respective piezoelectric element 106) the conductor bore 110 can be extended fully from the upper bone-facing surface 102d through the lower bone-facing surface 102e. In this instance, the piezoelectric element 106 could be embedded in the implant body 102 as described above, or could be configured to fit loosely in the conductor bore 110.

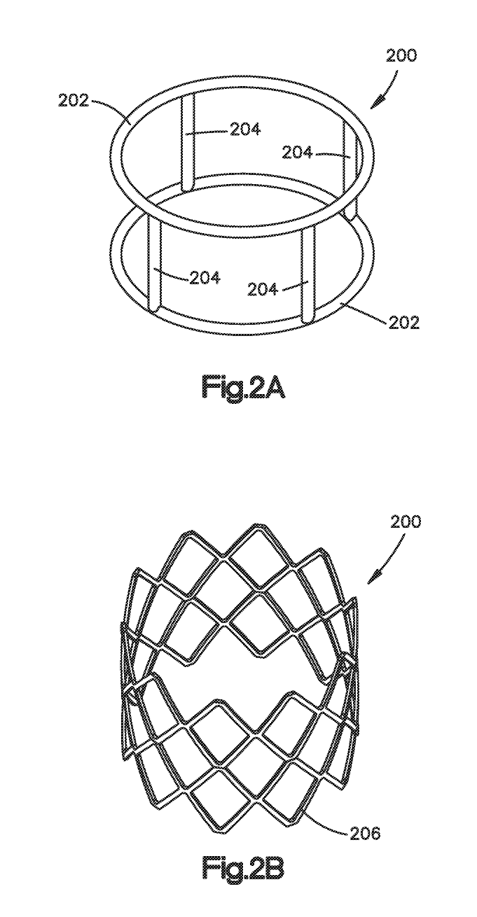

It should be appreciated that non-conducting elements (not shown) having the same or different geometries as the conductors 108 can be provided for use in the piezoelectric implant assembly 100. One or more of the non-conducting elements can be disposed into the conductor bores 110 in place of one or more corresponding conductors 108, for example to tune conduction of the signal emitted by the piezoelectric elements 106 or the piezoelectric frame 200 (see FIGS. 2A-B) to particular areas of the surrounding bones, tissues, and/or bodily fluids. Furthermore, the non-conducting elements can be configured to act as shielding, or insulating non-conducting elements that prevent direct contact between the piezoelectric elements 106 and neighboring bones, tissues, and/or bodily fluids.

The conductive engagement and resulting current flow between the piezoelectric elements 106 and the conductors 108 may occur via direct physical contact between the piezoelectric element engaging ends 108b of the conductors 108 and the respective upper and/or lower surfaces 106a-b of the piezoelectric elements 106. The physical contact may be established in accordance with how the piezoelectric elements 106 and the conductors 108 are embedded in the implant body 102, or the piezoelectric elements 106 and the conductors 108 may be glued, soldered, or otherwise affixed together along their respective interfaces. Alternatively, conductive engagement can be achieved by connecting the piezoelectric elements 106 to the conductors 108 utilizing one or more electrical leads, or the piezoelectric elements 106 and the conductors 108 may not be in physical contact and/or connected at all, but rather may be within an appropriate proximity, for example so as to induce electromagnetic effects.

Referring now to FIGS. 2A-B, one or more of the piezoelectric components, or elements, of the piezoelectric implant assembly 100 can be configured as a piezoelectric frame 200. The piezoelectric frame 200 can include one or more frame members that are formed partially or entirely of piezoelectric materials, such as those described above. For example, as illustrated in FIG. 2A, the piezoelectric frame 200 can include upper and lower frame members, such as frame rings 202. The frame rings 202 can be spaced apart along the transverse direction T by additional frame members, such as frame struts 204, the frame struts 204 extending between the upper and lower frame rings 202 along the transverse direction T, and coupled thereto using any appropriate connection method. Alternatively, the piezoelectric frame 200 may be configured as a piezoelectric lattice, as illustrated in FIG. 2B. The piezoelectric lattice may be constructed of a plurality of interconnected members, such as lattice section 206. Although the lattice sections 206 are depicted as having diamond shaped geometries, any other geometries can be used for the lattice sections 206 as desired.

It should be appreciated that although the piezoelectric frames 200 are depicted in FIGS. 2A-B as constructed of particular components, such as frame rings 202 coupled to frame struts 204 and interconnected lattice sections 206, that a piezoelectric frame 200 could be constructed using any combination of the above described or other frame elements as desired. It should further be appreciated that a piezoelectric frame 200 may be constructed using any combination of components made of piezoelectric materials and components made of non-piezoelectric materials, as desired.

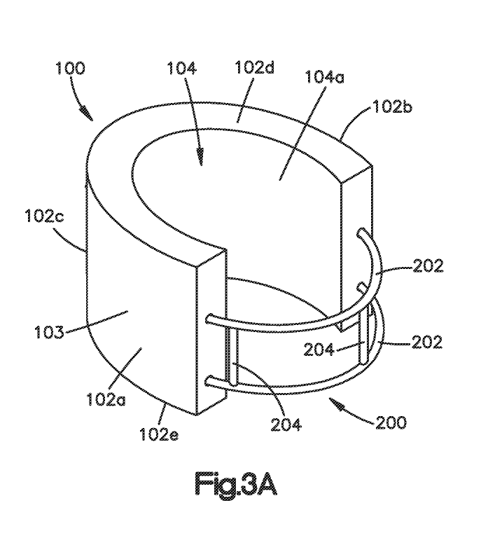

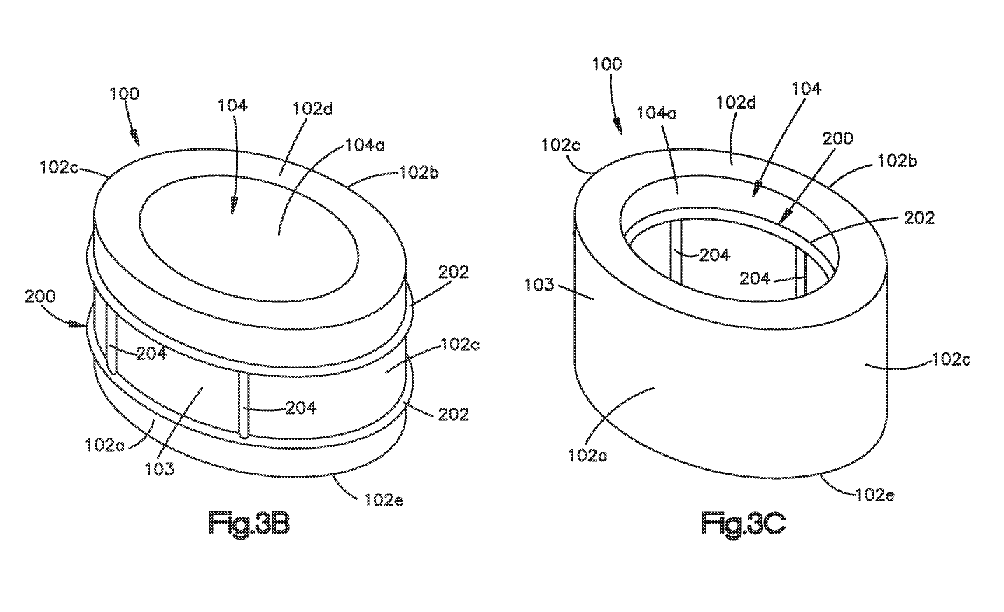

Referring now to FIGS. 3A-C, a piezoelectric frame 200 can be embedded in the implant body 102 in a number of ways. For example, the piezoelectric frame 200 can be completely embedded within the implant body 102, as depicted in FIG. 3A, for example by overmolding the implant body 102 over the piezoelectric frame 200. Alternatively, the piezoelectric frame 200 can be configured to embed in the side surface 103 of the implant body 102 along the contours of the anterior and posterior side 102a-b and the sides 102c (i.e., the side surface along the outer perimeter of the implant body 102), as illustrated in FIG. 3B, or can be configured to embed in the implant body 102 along the contour of the inner bore surface 104a of the implant bore 104, as illustrated in FIG. 3C. In operation, when an anatomical load is applied to the piezoelectric implant assembly 100, as described in more detail below, forces will be transferred from the implant body 102 to the piezoelectric frame 200. In response to the forces, the piezoelectric frame 200 will generate, or emit a signal in the form of an electromagnetic field into surrounding bone, tissues, and/or bodily fluids. In an alternative embodiment, one or more supplemental electrical components, for instance an inductor, battery, resistor, diode, capacitor, and the like can be embedded into the implant body 102. The supplemental electrical components can be configured to enhance, tune, or otherwise alter characteristics of the electromagnetic field emitted by the piezoelectric frame 200. Each of the one or more supplemental electrical components can be isolated from the piezoelectric frame 200, neighboring bone, tissues, and/or bodily fluids, or can be electrically and/or mechanically coupled to the piezoelectric frame 200, neighboring bone, tissues, and/or bodily fluids in one or more locations as desired. It should be appreciated that one or more supplemental electrical components can also be incorporated into any other configurations of the piezoelectric implant assembly 100 as appropriate.

It should be appreciated that embedding the piezoelectric frame 200 in the implant body 102, as depicted in FIGS. 3B-C, can include recessing the piezoelectric frame 200 into the respective surface of the implant body 102, or otherwise affixing the piezoelectric frame 200 to the respective surface of the implant body 102. It should further be appreciated that a piezoelectric frame 200 can be configured in combination with one or more conductors 108, non-conducting elements, and/or conductor bores 110, as described above. For example, one or more of the conductors 108 can be disposed in the implant body 102 so as to conductively engage with the piezoelectric frame 200 at one or more respective contact surfaces, or locations. The conductors 108 can be configured to emit and conduct an electric current from the piezoelectric frame 200 directly into surrounding bone, tissues, and/or bodily fluids. Moreover, it should be appreciated that although the piezoelectric implant assemblies 100 illustrated in FIGS. 3A-C each incorporate a single piezoelectric frame 200, that the piezoelectric implant assembly 100 should not be so limited, and that multiple piezoelectric frames 200 having any configuration, size, geometry, and the like can be incorporated within a single piezoelectric implant assembly 100.

Referring now to FIG. 1D, in operation the piezoelectric implant assembly 100 can be inserted between two adjacent vertebral bodies V1 and V2, for example after a total discectomy has been performed. The piezoelectric implant assembly 100 can be inserted such that the upper and lower bone-facing surfaces 102d-e engage with the inferior endplate of the vertebral body V1 and the superior endplate of the vertebral body V2, respectively. When the piezoelectric implant assembly 100 is subjected to anatomical loading and unloading, such as results from ambulation of the patient's body, compressive forces F act upon the piezoelectric implant assembly 100. In response to the compressive forces F, a potential difference develops between the superior and inferior surfaces 106a-b of each of the piezoelectric elements 106. The piezoelectric elements 106 are configured to generate, or emit a current in response to this potential difference that can be conducted from the piezoelectric elements 106 through the conductors 108 to respective endplates of the vertebral bodies V1 and V2. Alternatively, when the piezoelectric implant assembly 100 does not include the conductors 108, the piezoelectric elements 106 generate, or emit signals in the form of electromagnetic field in the area of the vertebral bodies V1 and V2 and neighboring tissues. It should be appreciated that although FIG. 1D depicts operation of the piezoelectric implant assembly 100 with the piezoelectric elements 106, that operation will be similar if one or more piezoelectric frames 200 are embedded in the implant body as a supplement to, or replacement for, the piezoelectric elements 106.

The electric current and/or magnetic fields emitted by the piezoelectric elements 106 can promote and/or enhance the naturally occurring osteoblastic activity in the surrounding bone, tissues, and/or bodily fluids, such as the endplates of the vertebral bodies V1 and V2, and in any bone growth inducing substances packed in the implant bore 104, resulting in increased bone growth and/or thickening. Therefore, it is desirable to locate the piezoelectric elements and/or piezoelectric frame 200 in appropriate proximity to the interface between the endplates of the vertebral bodies V1 and V2 and the bone growth inducing substances in the implant bore 104 so as to assist in fusion and bone remodeling between the vertebral bodies V1 and V2 and the bone growth inducing substances. Additionally, promotion and/or enhancement of osteoblastic activity may reduce the time required for fusion to occur between the adjacent vertebral bodies V1 and V2 and the piezoelectric implant assembly 100.

The natural cyclical loading and unloading experienced by a patient's musculoskeletal system, for example during ambulation and/or other activities, is well suited for application to the piezoelectric implant assembly 100, as a constant static loading of the piezoelectric implant assembly 100 and the adjacent vertebral bodies V1 and V2 might reduce blood flow to the areas in which bone growth is desired, reduce the consistency of piezoelectric signaling generated by the piezoelectric implant assembly 100, and may cause necrosis, as is often observed with bone plating systems that are too tightly lagged to an associated bone.

All or a portion of the electric current and/or electric potential generated by the piezoelectric elements 106 and/or the piezoelectric frame 200 can be captured and stored in a battery, capacitor, fuel cell, or the like coupled to the piezoelectric implant assembly 100, for later delivery to electrical devices such as a pacemaker, pain reduction stimulator, growing spinal rod, brain stimulator, or the like.

In an alternative embodiment of the piezoelectric implant assembly 100, the piezoelectric elements 106, the piezoelectric frame 200, and/or the conductors 108 may be omitted in favor of an implant body 102 with piezoelectric properties. For example, the implant body 102 may be constructed entirely from a piezoelectric material, such that the implant body 102 itself generates or emits an electric current and/or electromagnetic field in response to anatomical loading. In another alternative embodiment, the implant body 102 may be constructed from a mixture of materials, such as a non electrically conductive base material mixed with piezoelectric components comprising pieces of piezoelectric material, such as crystals or fragments of piezoelectric material. The distribution of the piezoelectric pieces within the implant body 102 could be controlled and localized as desired, or the distribution of the piezoelectric pieces within the implant body 102 could be random. The piezoelectric pieces could also be configured for mixture with bone cements either homogenously, randomly, in a biased manner, or in an oriented or directed manner as required. Increasing the piezoelectric characteristics of bone cement as a result of the inclusion of the piezoelectric pieces can reduce bone resorption and increase osteoblastic activity, for instance during vertebral or other bony structure augmentation and restoration. It should be appreciated that the above alternative embodiments are not limited to the implant body 102 as illustrated and described herein, but can also be applied to any other orthopedic, orthodontic, or other type of implant, bone plate, or the like, as desired. For example, a pair of piezoelectric bone plates disposed on opposing sides of a fracture may be partially or fully constructed from piezoelectric materials. The piezoelectric bone plates may further have electric leads connected therebetween, for example across the fracture site or crossing within the fracture itself.

In yet another alternative embodiment, piezoelectric components, such as the above-described piezoelectric pieces, can be configured for use in a piezoelectric coating, wrapping, or other integrated material. The piezoelectric coating could be applied to any or all surfaces of the implant body 102. In addition to use with the piezoelectric implant assembly 100, the piezoelectric coating may be appropriate for use with a range of orthopedic, orthodontic, or other types of implants, for example in areas where such implants may or may not be in direct physical contact with a bone surface, soft tissue or bodily fluids; such as the underside or inside of a bone plate or fixation plate for long bone fractures in the vicinity of the fracture where fusion is desired, the outer and/or inner surfaces of intramedullary nails, the outer and/or inner surfaces of dental or maxillofacial implants, the stem or innards of hip replacement components, the external or internal keels of total disc replacement implants, the inferior and superior surfaces of other total disc replacement implants, the exterior and/or interior surfaces of components of plating and/or screw constructs, the exterior and/or interior surfaces of bone screws and nails, and the like.

In yet another alternate embodiment, the piezoelectric components of the piezoelectric implant assembly 100 and the other various piezoelectric components described herein can be configured to be stimulated by receipt of an electromagnetic signal generated or emitted by an external device in the vicinity of the piezoelectric implant assembly 100 and the other various piezoelectric components. The received signal can induce a mechanical and/or electromagnetic interaction between the piezoelectric implant assembly 100 or various piezoelectric components and the surrounding bone, tissues, and/or bodily fluids.

It should be appreciated that components of the piezoelectric implant assembly 100 can be provided in a variety of piezoelectric implant kits. The components of the kits can be configured the same or differently. For example, within a single kit, implant bodies 102 can be provided having various sizes, shapes, piezoelectric components, can be provided with varying numbers and/or locations of conductor bores, and the like. The kits can also be configured differently with respect to which components are included in the kits. For example, kits can be provided having any combination of implant bodies 102, piezoelectric elements 106, conductors 108, non-conducting elements, and/or piezoelectric frames or frame components.

Although the components of the piezoelectric implant assembly 100 have been described herein with reference to preferred embodiments and/or preferred methods, it should be understood that the words which have been used herein are words of description and illustration, rather than words of limitation. For example, it should be appreciated that the piezoelectric elements 106 and/or the piezoelectric frame 200 can be configured using any geometries as desired, and further that the piezoelectric implant assembly 100 can be constructed using any combination of the piezoelectric elements 106, the piezoelectric frame 200, or the conductors 108. Furthermore, it should be appreciated that although the piezoelectric implant assembly 100 has been described herein with reference to particular structure, methods, and/or embodiments, the scope of the instant disclosure is not intended to be limited to those particulars, but rather is meant to extend to all structures, methods, and/or uses of the piezoelectric implant assembly 100. Those skilled in the relevant art, having the benefit of the teachings of this specification, may effect numerous modifications to the piezoelectric implant assembly 100 as described herein, and changes may be made without departing from the scope and spirit of the instant disclosure, for instance as recited in the appended claims.

What is claimed:

1. A piezoelectric implant assembly comprising:
an implant body extending along a transverse direction between opposing upper and lower bone-facing surfaces; and
a piezoelectric component embedded in the implant body such that the piezoelectric component does not contact surrounding bone,
wherein when the implant body is subjected to an anatomical load, the piezoelectric component emits a signal that stimulates growth in the surrounding bone.

2. A piezoelectric implant assembly as recited in claim 1, wherein the piezoelectric component is completely embedded within the implant body.

3. A piezoelectric implant assembly as recited in claim 2, wherein the piezoelectric component comprises a piezoelectric frame.

4. A piezoelectric implant assembly as recited in claim 3, wherein the signal emitted by the piezoelectric frame comprises an electromagnetic field.

5. A piezoelectric implant assembly as recited in claim 2, wherein a conductor bore extends into the implant body from one of the upper or lower bone-facing surfaces, the conductor bore having an interior end exposing a portion of the piezoelectric component, and
wherein the implant assembly further comprises a conductor disposed in the conductor bore, the conductor having opposing upper and lower ends, the lower end configured to engage the exposed portion of the piezoelectric component, and the upper end configured to engage the surrounding bone, and
wherein the signal emitted by the piezoelectric component comprises an electrical current conducted from the piezoelectric component into the surrounding bone via the conductor.

6. A piezoelectric implant assembly as recited in claim 1, wherein the piezoelectric component comprises a piezoelectric frame.

7. A piezoelectric implant assembly as recited in claim 6, wherein the implant body defines a side surface between the upper and lower bone-facing surfaces along a perimeter of the implant body, and
wherein the piezoelectric frame is embedded in the side surface.

8. A piezoelectric implant assembly as recited in claim 6, wherein an implant bore extends through the implant body from the upper bone-facing surface through the lower bone-facing surface, the implant bore defining an inner bore surface, and
wherein the piezoelectric frame is embedded in the inner bore surface.

9. A piezoelectric implant assembly as recited in claim 1, wherein the piezoelectric component likewise emits the signal when an external electromagnetic signal is received by the piezoelectric component.

10. A piezoelectric implant comprising an implant body extending along a transverse direction between opposing upper and lower bone-facing surfaces,
wherein application of an anatomical load to the implant body causes the implant body to stimulate growth in surrounding bone.

11. A piezoelectric implant as recited in claim 10, wherein the implant body is constructed of a mixture of materials, the mixture comprising:
a non electrically conductive base material; and
a plurality of piezoelectric pieces.

12. A piezoelectric implant as recited in claim 11, wherein the implant is constructed such that the plurality of piezoelectric pieces are randomly distributed throughout the implant body.

13. A piezoelectric implant as recited in claim 10, wherein the implant body is constructed entirely of piezoelectric material.

14. A piezoelectric implant as recited in claim 10, wherein the implant body defines a side surface along an outer perimeter of the implant body extending between the upper and lower bone-facing surfaces, and
wherein at least one of the upper and lower bone-facing surfaces and the side surface are coated with a piezoelectric coating.

15. A method of stimulating bone growth, the method comprising:
inserting a piezoelectric implant between adjacent bony surfaces, the piezoelectric implant comprising an implant body, and a piezoelectric component embedded in the implant body such that the piezoelectric component does not contact the adjacent bony surfaces,
wherein when the implant body is subjected to an anatomical load, the piezoelectric component emits a signal that stimulates bone growth in the adjacent bony surfaces.

16. A method as recited in claim 15, wherein the signal is an electric current, and
wherein the piezoelectric implant further comprises a conductor disposed in the implant body, the conductor establishing an electrical connection between the piezoelectric component and at least one of the adjacent bony surfaces, such that the electric current flows from the piezoelectric component through the conductor and into the at least one of the adjacent bony surfaces.

17. A method as recited in claim 15, wherein the piezoelectric component is completely embedded within the implant body, and
wherein the signal comprises an electromagnetic field.
